(19) United States
(12) Patent Application Publication
(10) Pub. No.: US 2026/0216522 A1
(43) Pub. Date: Jul. 30, 2026

(54) Fixation Sleeve and Assembly Comprising Such Sleeve

(71) Applicant: BIOTRONIK SE & Co. KG, Berlin (DE)

(72) Inventor: Roland Eichberg, Berlin (DE)

(73) Assignee: BIOTRONIK SE & Co. KG, Berlin (DE)

(21) Appl. No.: 19/158,244

(22) PCT Filed: Feb. 12, 2024
(86) PCT No.: PCT/EP2024/053436

§ 371 Date: Aug. 20, 2025

(30) Foreign Application Priority Data
Mar. 2, 2023 (EP) 23159599.2
Mar. 23, 2023 (EP) 23163710.9

(51) Int. Cl. A61N 1/375

(52) U.S. Cl. CPC A61N 1/37518

(57) ABSTRACT

A fixation sleeve for an implantable lead including a body forming a wall and an inner lumen configured to receive an implantable lead. An outer surface of the body includes at least one guide element configured to receive and clamp a thread and/or the outer surface of the body includes in circumferential direction at least one first segment and at least one second segment, wherein the segments are configured to be wrapped by a thread. Further, a respective assembly including such fixation sleeve and a lead and/or a thread is described.

### CROSS-REFERENCE TO RELATED APPLICATIONS

This application is the United States National Phase under 35 U.S.C. § 371 of PCT International Patent Application No. PCT/EP2024/053436, filed on Feb. 12, 2024, which claims the benefit of European Patent Application No. 23163710.9, filed on Mar. 23, 2023, and European Patent Application No. 23159599.2, filed on Mar. 2, 2023, the disclosures of which are hereby incorporated by reference herein in their entireties.

### TECHNICAL FIELD

The invention relates generally to a fixation sleeve for an implantable (medical device) lead and an assembly comprising the (medical device) lead and the fixation sleeve.

### BACKGROUND

Medical devices such as cardiac pacemakers or defibrillators comprise at least one lead emanating from the medical device and terminate at a predefined treatment location on the tissue. The lead applies electrical pulses to the predefined treatment location and/or transmits electrical signals detected at the predefined tissue location to the medical device. A fixation sleeve is used to secure the implanted lead near the treatment location at a predefined fixation location, in particular at the point of entry into a blood vessel leading to the heart. Such fixation sleeve is generally configured as tubular member, the cavity or lumen of which is adapted to sheathe the (electrically conductive) lead.

During implantation the lead is sheathed within the fixation sleeve. Once the lead is properly positioned at the predefined location at the patient's body, the fixation sleeve is slid down the lead to a point where it is supposed to be fixed at the patient's body—the fixation location. There it is wrapped with a thread. The thread is pulled tight and tied by the health care practitioner (HCP, e.g. clinician or nurse) to secure the fixation sleeve to the lead body so that the lead does not move in longitudinal direction with regard to the fixation sleeve. Additionally, the fixation sleeve is secured by the thread to the body tissue at the fixation location. Securing the fixation sleeve in this manner is important to provide permanent hemostasis and/or lead stabilization at the predefined fixing location.

However, to attach the fixation sleeve to the lead, the thread must be tightened and knotted (at least with a double knot). The problem here is that the first knot may come loose again while the second knot is being created (the “missing third finger” problem).

Furthermore, because a fixation sleeve is often constructed of soft, pliable material, problems may occur during the above-described procedure or afterwards. On the one hand, if the HCP pulls the thread too tight when securing the sleeve to the lead, the thread may cut through (at least parts of) the soft material of the fixation sleeve, and thereby possibly damaging the lead. When this happens, the lead must be replaced. Unfortunately, damage to the lead is often not detected until after the implantation is complete. Accordingly, an additional surgery is required to fix the problem thereby ultimately increasing the total cost of the implantation procedure. On the other hand, there may be problems if the fixation sleeve is not securely fixed to the lead body and the lead moves relative to the fixation sleeve within the patient's body.

Accordingly, there is a need for a fixation sleeve that solves at least of the above-mentioned problems, in particular solving the “missing third finger” problem, and/or reducing the risk of a thread cutting through the sleeve and thus possibly damaging and/or not properly fixing the lead.

The present disclosure is directed toward overcoming one or more of the above-mentioned problems, though not necessarily limited to embodiments that do.

### SUMMARY

The above object is solved by a fixation sleeve with the features of claims 1 and/or 7 as well as by an assembly comprising a lead and a fixation sleeve and/or comprising a thread and a fixation sleeve with the features of claims 14 and/or 15.

In particular, the above object is solved by a fixation sleeve for an implantable lead (e.g. an electrode lead) comprising a body forming a wall and an inner lumen configured to receive an implantable lead (or lead body), wherein an outer (shell) surface of the body comprises at least one guide element (e.g. a circumferential groove) configured to receive (and at least partially enclose) a thread (which encircles the body of the fixation sleeve), wherein the guide element is configured to clamp the thread. Preferably the guide element comprises at least one clamping section, wherein the width of (the cross section of) the (at least one) clamping section is dimensioned such that a clamping force is applied to the thread. Preferably the (at least one) clamping section is dimensioned such that the clamping force is applied to the thread, when the thread is tightened to fix the implantable lead in the fixation sleeve. Due to the guide element with the at least one clamping section, the thread is already held by the clamping section after a first knot is tied, and the first knot does not untie while a second knot is tied or created (“missing third finger” problem solved).

In one embodiment, the width of (the cross section of) the (at least one) clamping section is less than or equal to the diameter of the thread.

In one embodiment, the (pre-defined) diameter of the thread may be, for example, 0.1 to 0.8 mm, preferably 0.25 to 0.4 mm, particularly preferably 0.3 mm. The diameter of the thread may be up to twice as large as the width of the guide element, especially up to twice as large as the width (of the cross section) of the at least one the clamping section. In other words, the width (d) of the clamping section is at least half as large as the diameter (D) of the thread.

Preferably, the diameter of the thread is 10 to 20 percent larger, preferably 15 percent larger, than the width of (the cross section of) the guide element, especially the width (of the cross section) of the at least one clamping section. In other words, the width of (the cross section of) the guide element (or the width of the at least one clamping section) is 9 to 17 percent lower, preferably 13 percent lower, than the diameter of the thread.

The width of the clamping section may be, for example, 0.05 to 0,75 mm, preferably 0.05 to 0.45 mm, preferably 0.1 to 0.4 mm, preferably 0.13 to 0.36 mm, particularly preferably 0.25 mm. Preferably, the width of the clamping section is 0.25 mm if the diameter of the thread is 0.3 mm, the width of the clamping section is 0.2 mm if the diameter of the thread is 0.25 mm, the width of the clamping section is 0,3 mm if the diameter of the thread is 0.35 mm, or the width of the clamping section is 0,34 mm if the diameter of the thread is 0.4 mm.

In cross section the guide element 65 (or the groove) may have the shape of a rectangle, a funnel, a triangle, an incomplete circle, a cone, or a truncated cone (in each case upwards or outwards open).

The above fixation sleeve has a body (e.g. a tubular body) that forms an outer (shell) surface, an inner surface surrounding the inner lumen and a wall between the inner surface and the outer surface. The inner lumen is a through hole for receiving the lead that may be formed as a cylindrical lumen. The longitudinal axis of the lumen (and the lead when accommodated within the lumen) is referred to as the longitudinal direction. The radial and circumferential directions refer to this longitudinal direction.

In a preferred embodiment, the guide element is a circumferential groove, preferably with the clamping section extending along the entire groove. The circumferential groove arranged on or in the outer surface for the thread may extend along part of or along the entire circumference of the outer surface. A groove root of the circumferential groove having, for example, a (constant or varying) radial distance to the longitudinal axis of the lumen.

In one embodiment of the invention, the guide element (e.g. the circumferential groove) comprises at least one reduced diameter section along its longitudinal extension, wherein an inner diameter of the cross section of at least one reduced diameter section of the guide element is less than or equal to 2 times the diameter of the thread that is usually used for suturing the fixation sleeve to the patient's body tissue. The longitudinal extension of the guide element (e.g. the circumferential groove) may be the extension along the groove and runs therefore circumferential with regard to the longitudinal direction of the fixation sleeve. The inner diameter of the guide element may be measured perpendicular to the longitudinal extension of the guide element as the greatest distance of the opposite inner walls. In one embodiment, the inner diameter of the reduced diameter section is equal to or greater than the thread's diameter. If the inner diameter of the reduced diameter section of the guide element is dimensioned in this way, there the guide element grips or clamps the knot after the first half of the knot is tied. This eases the knot tying by the HCP. The HCP may use the surgeon's knot or the reef knot, for example. Accordingly, the fixation sleeve may be securely fixed by the HCP to the lead (and the patient's body).

The above problem is further solved by a fixation sleeve for an implantable lead comprising a body forming a wall and an inner lumen configured to receive an implantable lead, wherein the body comprises in circumferential direction at least one first segment and at least one second segment, wherein the segments (or the first and the second segment) are configured to be wrapped by a thread (e.g. a ligature thread), and wherein the segments are attached to the body such that the at least one first segment or the at least one second segment is pressed against the implantable lead when the thread is tightened to fix (or attach) the implantable lead in the fixation sleeve.

In one embodiment, at least one of the (at least one first and at least one second) segments (extending into longitudinal direction of the implantable lead and) comprising the at least one guide element (as described above).

In one embodiment, the guide element further comprises at least one guiding section, and the at least one clamping section of the guide element is formed by the at least one second segment and/or at least one guiding section of the guide element is formed by the at least one first segment, wherein the width of (the cross section of) the at least one guiding section is greater than the diameter of the thread. Thus, the at least one guiding section does not apply any clamping force on the thread.

The fixation sleeve may comprise two or more guide elements (e.g. circumferential grooves) for the thread (or two or more threads).

If there are two or more first and second segments, the guiding section and the clamping section of the guide element (e.g. the circumferential groove) may form several sub-sections.

The above fixation sleeve may have analogous properties as the above-described fixation sleeve with regard to its (tubular) body and the guide element. Additionally, it comprises at least one first segment and at least one second segment, wherein with regard to the first and second segments, the above-described properties of these segments apply.

Additionally, the at least one second segment may comprise means for attaching the sleeve to the lead body, for example provided by its elasticity.

In one embodiment, the elasticity of the material of the at least one second segment is greater than the elasticity of the at least one first segment and the wall thickness of the at least one second segment is greater than the wall thickness of the at least one first segment. This feature has the effect that the material of the at least one second segment is deformed elastically to a greater extent (or deformed more elastically) than the material of the at least one first segment when the thread accommodated within the guide element (e.g. the circumferential groove) is pulled tight. This tension is transmitted to the inner wall within the respective second segment so that this inner wall provides a compressive force that acts on the lead extending within the inner lumen. Thereby, the inner wall of the respective second segment of the fixation sleeve is pressed onto the outer surface of the lead so that the lead is securely attached to the fixation sleeve and cannot move in longitudinal direction.

In one embodiment of the fixation sleeve, in particular if each first segment and each second segment extend over the entire wall thickness, the hardness of the material of the at least one first segment is greater than the hardness of the material of the at least one second segment. For example, the material of the first segment may be one material of the group comprising a thermoplastic material, e.g. Polyether ether ketone (PEEK) or Polyurethane (PU). For example, the material of the second segment may be a soft and/or elastic material comprising or being silicone. The at least one first segment reinforces the wall of the respective portion of the tubular body such that the thread (also called suture) does not indent and abrases the wall at the first segment as easy as in the second segment. The at least one first segment hinders the penetration of the thread into the (tubular) wall and acts like a stop member when the thread is pulled tight. Accordingly, the risk is reduced that the thread accommodated within the at least one guide element (e.g. circumferential groove) cuts through the material and damages the lead accommodated within the inner lumen of the fixation sleeve.

In one embodiment, the at least one first segment and the at least one second segment are arranged alternating in circumferential direction. Accordingly, one first segment is located next to one or two second segments and vice versa in circumferential direction. By the alternate arrangement of first and second segments, the radial forces provided by the thread during and after it is pulled tight is equally distributed over the whole circumference.

The production cost of the fixation sleeve can be reduced if the fixation sleeve comprises at least one first (tubular) element and at least one second (tubular) element, wherein the first element forms at least one first segment and the at least one second element forms at least one second segment and wherein the at least one first element and the at least one second element are separately manufactured. The at least one first element is attached to the at least one second element. The separate manufacturing of the elements and the subsequent attachment eases the production process with the tradeoff of increased number of production steps. For example, the at least one first element and the at least one second element is produced by injection molding. The attachment of one first element and one second element may be provided by a positive locking connection, for example, a snap fit connection, wherein the male part may be provided by the first element and the female part by the second element or vice versa. Alternatively, the attachment may be provided by a frictional connection or a connection using a glue. For example, the fixation sleeve (also called suture sleeve) may comprise one first element and two second elements, each of which is attached to one end portion formed on both longitudinal ends of the first element. In this example, if viewed in the longitudinal direction the first element may be located in a central section of the fixation sleeve and each one of the two second elements are located in an end section of the fixation sleeve.

In one embodiment, the first segment is formed by a longitudinally extending first finger or web (bar, land) of the first element and the second segment is formed by a longitudinally extending second finger or web (bar, land) of the second element, wherein the first finger or web and the second finger or web mesh with one another. The at least one first finger of the first element may longitudinally extend from a tubular body section of the first element and the at least one second finger of the second element may longitudinally extend from a tubular body section of the second element. With regard to the embodiment that the at least one finger is formed as a web (bar, land) the first web may longitudinally extend between two tubular body sections of the first element connecting both body sections. Similarly, with regard to the embodiment that the at least one finger is formed as a web (bar, land) the second web may longitudinally extend between two (tubular) body sections of the second element connecting both body sections. When the first element and the second element are attached to each other, the at least one first finger or web meshes with the at least one second finger or web so that all fingers adjoining each other in the circumferential direction form the whole circumference of the portion of the tubular body that comprises the at least one guide element (e.g. circumferential groove). For example, the fixation sleeve may comprise one first element with four webs at each longitudinal end and two second elements each of which has four fingers at one of their ends. The four webs of one end of the first element mesh with the four fingers of one second element.

In one embodiment, the first element may comprise at least one eyelet at its outer (shell) surface. The eyelet may be used to thread the fixation sleeve to the patient's tissue at the fixation location. The inner hole diameter of such eyelet may be, for example, 1 mm. Providing the eyelet at the harder first element rather than at the second element has the advantage, that the risk is reduced, as well, that such eyelet cuts through the eyelet rim.

In one embodiment, the outer (shell) surface of the at least one first segment and/or of the at least one second segment adjacent to the at least one guide element is inclined towards the guide element. This inclination further eases the accommodation of the thread within the guide element as it helps the thread to slide towards the guide element during implantation procedure. Accordingly, the HCP work is further expedited.

All above-described embodiments and features of the fixation sleeve may be combined with each other, in particular all embodiments of the fixation sleeve comprising the guide element with all embodiments of the fixation sleeve comprising the first segment and the second segment.

The above object is further solved by an assembly comprising a thread, for example a ligature thread, having a pre-defined diameter and a fixation sleeve as described above. The assembly with the fixation sleeve and the thread has the above-described advantages and embodiments (with regard to the fixation sleeve). The thread may comprise or consist of mersilene. The (pre-defined) diameter of the thread may be, for example, 0.1 to 0.8 mm, preferably 0.25 to 0.4 mm, particularly preferably 0.3 mm. The difference between the width of the guide element, especially the width (of the cross section) of the at least one clamping section, and the diameter of the thread should be selected depending on the material and hardness of the fixation sleeve, in particular the part of the fixation sleeve forming the clamping section. The thread may be up to twice as wide (or the diameter of the thread may be up to twice as large) as the width of the guide element, especially the width (of the cross section) of the at least one the clamping section. Preferably, the thread is 10 to 20 percent wider than the width of the guide element, especially the width (of the cross section) of the at least one the clamping section.

The above object is further solved by an assembly comprising a lead for a medical device (such as a pacemaker, a defibrillator, a cardiac monitor, or a loop recorder) and a fixation sleeve as described above. The fixation sleeve may sheathe a body section of the lead and may be movable along the lead (or the lead body) in longitudinal direction prior implantation. Furthermore, the assembly may further comprise a thread having a pre-defined diameter (as defined above).

The assembly with the fixation sleeve and the lead (and the thread) has the above-described advantages and embodiments. Prior implantation and prior fixation of the fixation sleeve to the lead and the patient's tissue, the fixation sleeve can be moved along the lead (or the lead body) in the longitudinal direction to adapt the position of the fixation sleeve to the personal needs of the patient. During implantation procedure, first, the tip (electrode) of the lead may be properly placed at the predefined treatment position. Then, if necessary, after sliding the fixation sleeve along the lead (or the lead body), the fixation sleeve is attached to the fixation location. Usually, the lead comprises an electrically conducting tip at its distal end and a connector for electrical and mechanical connection to the medical device at its proximal end.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawings, wherein

FIG. 1 shows a patient with an example medical device, lead and fixation sleeve in a front view,

FIG. 2 depicts a first embodiment of a fixation sleeve with a lead section in a perspective side view,

FIG. 3 depicts a second embodiment of a fixation sleeve with a lead section in a perspective side view,

FIG. 4 depicts a third embodiment of a fixation sleeve with a lead section in a perspective side view,

FIG. 5 shows the embodiment of the fixation sleeve of FIG. 4 in a longitudinal section, an enlarged detail A of this section, an enlarged cross section of a thread and as well as the enlarged detail A with a thread (detail A'),

FIG. 6 depicts a first element of the embodiment of the fixation sleeve of FIG. 4 in a perspective side view,

FIG. 7 shows a second element and a third element of the embodiment of the fixation sleeve of FIG. 4 in a perspective side view,

FIG. 8 depicts a fourth embodiment of a fixation sleeve with a lead section in a perspective side view,

FIG. 9 shows the embodiment of the fixation sleeve of FIG. 8 in a longitudinal section,

FIG. 10 depicts a fifth embodiment of a fixation sleeve with a lead section in a perspective side view, and

FIG. 11 shows the embodiment of the fixation sleeve of FIG. 10 in a longitudinal section.

### DETAILED DESCRIPTION

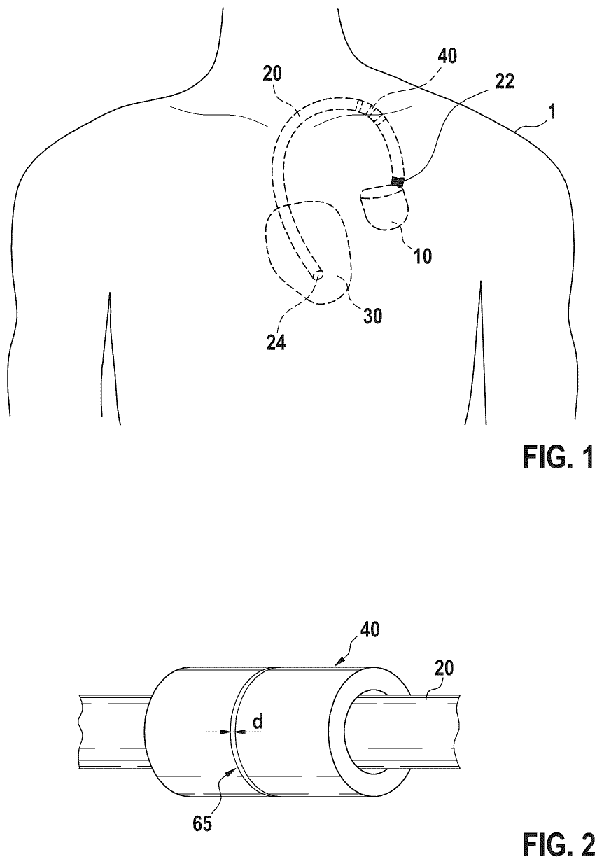

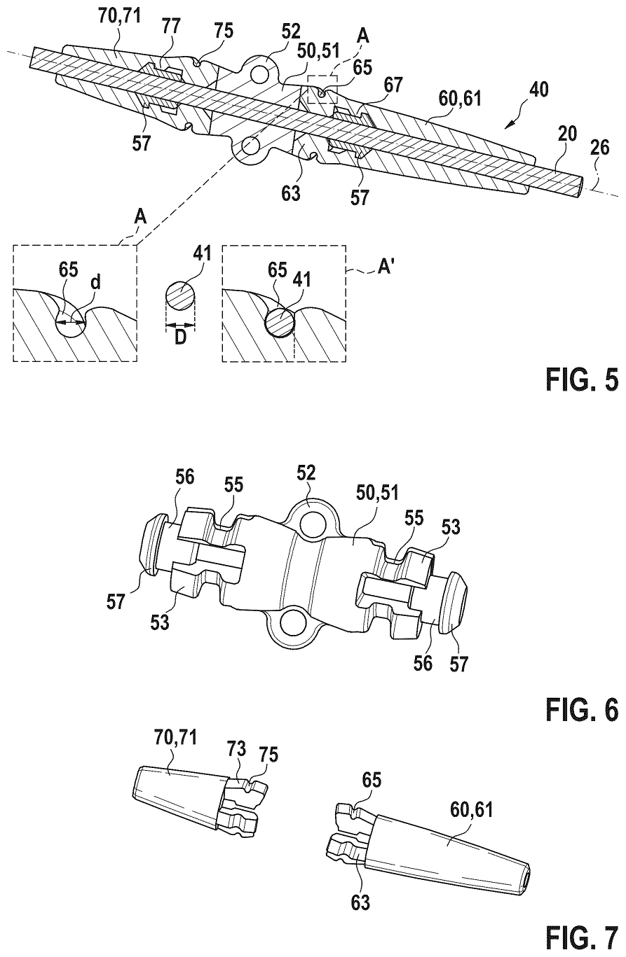

Referring to the drawings, FIG. 1 shows a pacemaker system including a pacemaker 10 of a patient 1, an electrode lead 20 electrically and mechanically connected to the pacemaker 10 by a connector 22. A distal tip of the lead 20 with an electrode 24 is positioned within a heart 30 of the patient 1 at a predefined treatment position. The lead 20 passes through a fixation sleeve 40 which in turn is sutured into surrounding tissue of the patient 1 at a predefined fixation position using a thread 41 (e.g. as shown in FIG. 5) to help position and stabilize the location of lead 20 (e.g. at the point of entry into a blood vessel leading to the heart).

FIG. 2 to 7 show details of three embodiments of a fixation sleeve 40 with the lead 20 extending through the inner lumen of the fixation sleeve 40. The fixation sleeve 40 forms an assembly together with the lead 20 (and/or with the thread 41).

FIG. 2 shows a first embodiment of a fixation sleeve 40 comprising a tubular body forming a wall and an inner lumen to receive an implantable lead 20. The outer surface of the tubular body comprises a guide element 65, which is (shaped as) a circumferential groove. This guide element 65 is configured to receive a thread, in particular a ligature thread. The guide element 65 may receive and at least partially enclose the thread. The guide element 65 receives the thread (and encloses the thread completely or partially) when the thread is wrapped around the fixation sleeve 40 or when the thread encircles the fixation sleeve 40. The guide element 65 (in the embodiment of the fixation sleeve in FIG. 2) comprises one clamping section, more precisely, the guide element 65 in the form of a circumferential groove is completely formed as a clamping section. The clamping section (and thus the complete guide element 65) is dimensioned such that a clamping force is applied to the thread 41, when the thread is tightened to fix the implantable lead 20 in the fixation sleeve 40. Of course, the guide element 65 (or groove) could also comprise two or more clamping sections, which would alternate with wider guiding sections (which do not apply any clamping force on the thread) in the circumferential direction. The width d of (the cross section of) the at least one clamping section is less than the diameter of the thread. Preferably, the width d is 0.25 mm, in particular if the diameter of the thread is 0.3 mm. In cross section the guide element 65 (or the groove) may have the shape of a rectangle, a funnel, a triangle, an incomplete circle (as indicated or approximated in FIG. 5), a cone, or a truncated cone (in each case upwards or outwards open).

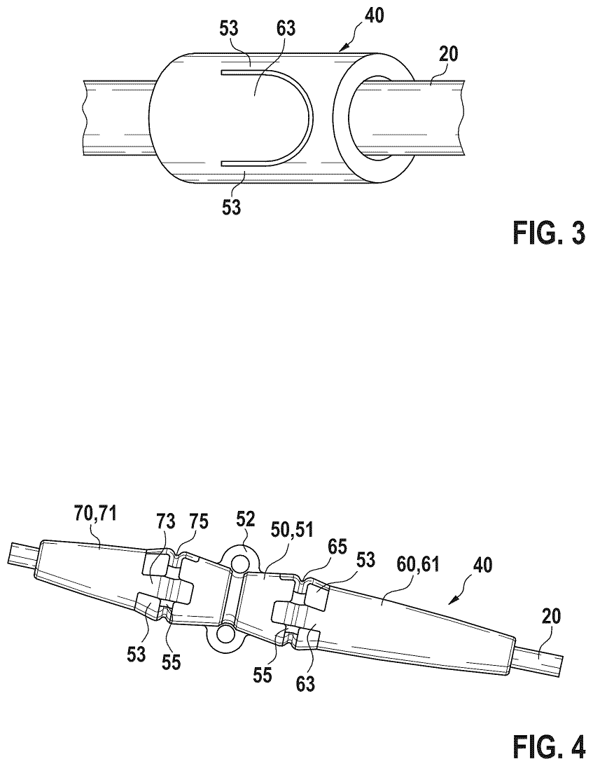

FIG. 3 shows a second embodiment of a fixation sleeve 40 comprising a tubular body forming a wall and an inner lumen to receive an implantable lead 20. The body of the fixation sleeve 40 comprises (in circumferential direction) one first segment 53 and one second segment 63. Both segments 53, 63 are configured to be wrapped by a thread, in particular a ligature thread. Further, the segments 53, 63 are attached to the body such that the second segment 63 is pressed against the implantable lead 20 when the thread is tightened to fix the implantable lead 20 in the fixation sleeve 40. In this particular embodiment of FIG. 3, both segments 53, 63 are an integral part of the body, i.e. they are formed in one piece. The second segment 63 forms a kind of tongue which has been cut into the body. When the thread encloses both segments and is tightened, the circumference of the body is reduced by pressing the second segment 63 onto the implantable lead 20. The pressing effect or pressing force on the implantable lead 20 may be enhanced by increasing the thickness of (the wall of the body in the area of) the second element 63, for example by applying a reinforcing element or thickening element to the outer surface of the second segment 63).

The embodiments of FIG. 2 and FIG. 3 may be combined and extended, as shown in the following figures (see FIGS. 4 to 11). Furthermore, any of the additional features shown in the following FIGS. 4 to 11 may be added to the first two embodiments of FIG. 2 and FIG. 3.

FIG. 4 shows a third embodiment of a fixation sleeve 40 comprising a first tubular element 50, a second tubular element 60 and a third tubular element 70, wherein the first tubular element 50 is located in between the second tubular 60 element and the third tubular element 70 along the longitudinal axis 26 of the electrode lead 20 or the inner lumen. The hardness of the material of the first element 50 is greater than the hardness of the material of the second element 60 and of the third element 70. Additionally, the elasticity of the material of the second element 60 and of the third element 70 is greater than the elasticity of the material of the first element 50. For example, the first element 50 is made of PEEK. The second element 60 and the third element 70 may consist of silicone. The first element 50, the second element 60 and the third element 70 are manufactured separately and are then assembled by snap fit connections as described below. The first element 50 forms the “first element” defined in the general part of the description above whereas the second element 60 and the third element 70 form “second elements” as defined in the general part of the description above.

The first element 50 which is shown in more detail in FIG. 6 comprises a tubular central section 51 with two eyelets 52 configured to receive a thread for attachment of the fixation sleeve 40 to the surrounding tissue at the fixation location. Further, the first element 50 comprises eight longitudinally extending webs 53. Four webs 53 project from each end (in longitudinal direction) of the central section 51 and form first segments of two tubular portions of the fixation sleeve 40 as defined in the general part of the description above. Each web 53 comprises a greater diameter section 55 (guiding section) of a guide element, here a circumferential groove for the thread. Further, the webs 53 are connected to another tubular section 56 at their longitudinal end that is opposite the central section 51. Each such other section 56 terminates at a section having the form of a tubular, truncated cone 57 that forms a snap fit connection with the second element 60 and the third element 70, respectively. The other tubular section 56 has a smaller wall thickness than the cone section 57. In addition, “guide element” is in the following (mainly) called “circumferential groove” or just “groove”.

The second element 60 and the third element 70 are similar elements but with a different dimension in longitudinal direction, i.e. the second element 60 is longer than the third element 70. The elements 60, 70 are shown in more detail in FIG. 7. Both elements 60, 70 comprise four fingers 63, 73 at one end which extend in longitudinal direction from a tubular section 61, 71 that form second segments of two tubular portions of the fixation sleeve sleeve 40 as described in the general part of the description above. Additionally, the tubular section 61, 71 has a slightly inclined shell surface such that its wall thickness reduces from the respective end that is opposite the fingers 63, 73. Thereby, the transition to the outer diameter of the lead body is smoothed.

Each finger 63, 73 comprises a reduced diameter section 65, 75 (clamping section) of the circumferential groove for the thread. Accordingly, a first circumferential groove is formed by the first element 50 and the second element 60 by the greater diameter sections 55 and the reduced diameter sections 65 and a second circumferential groove is formed by the first element 50 and the third element 70 by the greater diameter sections 55 and the reduced diameter sections 75 when the first element 50, the second element 60 and the third element 70 are connected by a snap fit connection. The snap fit connection is provided between the first element 50 and the second element 60 by the (harder, less elastic) cone 57 and an undercut 67 formed at the inner surface of the second element 60 (see FIG. 5). Similarly, another snap fit connection is provided between the first element 50 and the third element 70 by the (harder, less elastic) cone 57 and an undercut 77 formed at the inner surface of the third element 70. When the respective snap fit connection is established, the webs 53 mesh with the fingers 65 and 75, respectively, so that the full circumferential groove 55, 65 and 55, 75, respectively, is established and the full tubular body of the fixation sleeve 40 is assembled. The reduced diameter sections 67, 75 of the circumferential groove have an inner diameter d (perpendicular to the circumferential direction) that is equal to or less than 2 times the diameter D of the thread 41 that is to be accommodated within the groove (see FIG. 5 in detail A and A′, wherein detail A′ shows the reduced diameter section 65 of the groove with the thread 41). The same applies similarly to the reduced diameter section 75 of the fingers 73 of the third element 70. By the reduced diameter sections 65, 75 a knot within the thread 41 is gripped after the first half of the knot is tied. In contrast, the greater diameter sections 55 of the fingers 53 of the first element 50 ease knotting and accommodating of the thread 41 within the groove due to their greater diameter. For example, for a thread 41 with a diameter D of 0.3 mm, the reduced diameter sections 65, 75 (clamping sections) of the groove of the second element 60 and the third element 70, respectively, have an inner diameter d or a width d of 0.25 mm and the greater diameter sections 55 (guiding sections) of the groove of the first element 50 have an inner diameter or a width of 0.4 mm.

The wall thickness (in radial direction) at the groove sections 55 of the first element 50 is smaller than the wall thickness at the reduced parameter groove sections 65 of the second element 60. Similarly, the wall thickness (in radial direction) at the groove sections 55 of the first element 50 is smaller than the wall thickness at the reduced parameter groove sections 75 of the third element 70. Due to the higher elasticity of the second element 60 and the third element 70, a radial compressive force is provided in the respective section 65, 75 to the lead body of the lead 20 located within the inner lumen of the fixation sleeve 40 when a thread extending along the circumferential groove 55, 65 or 55, 75 is pulled tight. Thereby, the lead body is clamped and fixed to the fixation sleeve 40. On the other hand, the groove sections 55 formed by hard material act like stop members that prevent the thread to penetrate the circumferential groove so that the thread is hindered cutting through the wall of the fixation sleeve 40 and damaging the lead body.

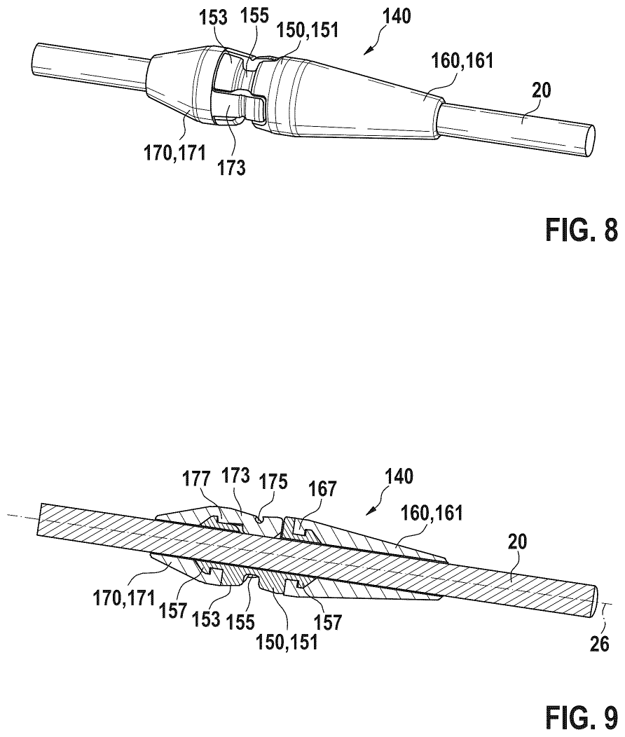

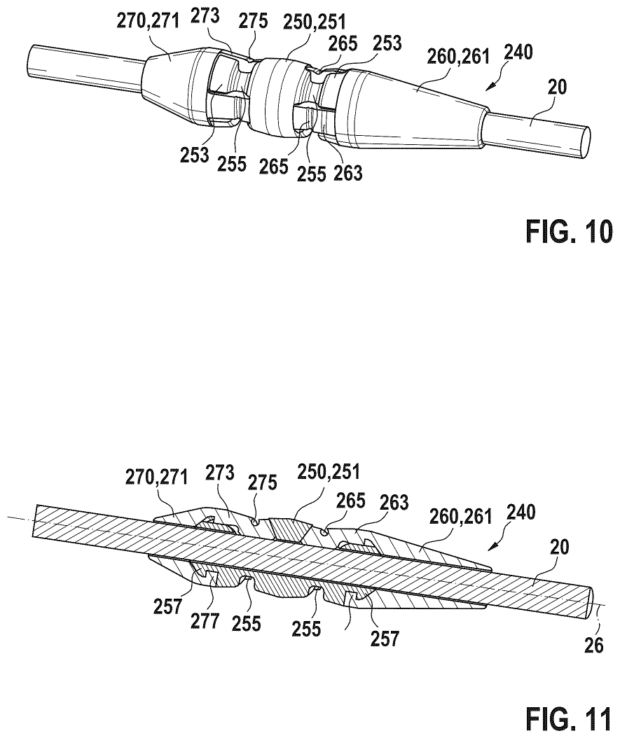

FIGS. 8 and 9 show a fourth embodiment 140 of a fixation sleeve and FIGS. 10 and 11 a fifth embodiment 240. Similar features of the fixation sleeve 40 and the fixation sleeves 140, 240 are designated with the same reference number in the ones and tens digits. They only differ by 100 (for the fixation sleeve 140) or by 200 (for the fixation sleeve 240) from the reference numbers of the first embodiment.

The fixation sleeve 140 differs from the first embodiment in that the first element 150 does not comprise any eyelet. Additionally, the third element 170 and the first element 150 only provide one circumferential groove for the thread that is formed by the greater diameter sections 155 of the webs 153 of the first element 150 and the reduced diameter sections 175 of the fingers 173 of the third element 170. The second element 160 does not form any circumferential groove for the thread. It acts like a cap of the first element 150 on one longitudinal end of this element. Similar to the first embodiment, the second element 160 forms a snap fit connection with the first element by a truncated cone 157 of the first element 150 and an undercut 167 of the second element 160. It is noted that the shell surface at the webs 153 and fingers 173 are inclined towards the circumferential groove 155, 175 so that the wall thickness is reduced into the (longitudinal) direction of the groove to support the accommodation of the thread within the groove 155, 175. The fixation sleeve 240 is similar to the first embodiment of a fixation sleeve 40 but does not comprise any eyelets at its first element 250. As one can derive from FIGS. 10 and 11, the shell surface of the central section 251 of the first element 250 as well as of the webs 253 and of the fingers 263, 273 of the second element 260 and the third element 270, respectively, is inclined towards the respective circumferential groove 255, 265 or 255, 275. Similar to the second embodiment of the fixation sleeve 140 by such surface structure the thread more easily slides towards the respective groove 255, 265 or 255, 275 and thereby work of the HCP is facilitated.

As one can derive from FIGS. 5, 9, 11 the webs 53, 153, 253 extend over the entire wall thickness in the tubular portion around the respective circumferential groove section 55, 155, 255. The same applies to the fingers 63, 73, 173, 263, 273 in the tubular portion around the respective circumferential groove section 65, 75, 165, 265, 275. The tubular portions defined in the above general part of the description are formed by the webs 53 and the fingers 63 as well as the webs 53 and fingers 73 around the respective circumferential groove of the fixation sleeve 40, by the webs 153 and the fingers 173 around the single circumferential groove of the fixation sleeve 140 and by the webs 253 and the fingers 263 as well as the webs 253 and fingers 273 around the respective circumferential groove of the fixation sleeve 240.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A fixation sleeve for an implantable lead comprising a body forming a wall and an inner lumen configured to receive an implantable lead, wherein an outer surface of the body comprises at least one guide element configured to receive and clamp a thread.

2. The fixation sleeve of claim 1, wherein the guide element comprises at least one clamping section, wherein the width of the at least one clamping section is dimensioned such that a clamping force is applied to the thread, when the thread is tightened to fix the implantable lead in the fixation sleeve.

3. The fixation sleeve of claim 2, wherein the width of the clamping section is less than or equal to the diameter of the thread.

4. The fixation sleeve of claim 2, wherein the width of the clamping section is at least half as large as the diameter of the thread.

5. The fixation sleeve of claim 2, wherein the width of the clamping section is 9 to 17 percent lower, preferably 13 percent lower, than the diameter of the thread.

6. The fixation sleeve of claim 2, wherein the width of the clamping section is 0.05 to 0,75 mm, preferably 0.13 to 0.36 mm, particularly preferably 0.25 mm.

7. A fixation sleeve for an implantable lead comprising a body forming a wall and an inner lumen configured to receive an implantable lead, wherein the body comprises in circumferential direction at least one first segment and at least one second segment, wherein the segments are configured to be wrapped by a thread, and wherein the segments are attached to the body such that the at least one first segment or the at least one second segment is pressed against the implantable lead when the thread is tightened to fix the implantable lead in the fixation sleeve.

8. The fixation sleeve of claim 7, wherein at least one of the segments comprising at least one guide element.

9. The fixation sleeve of claim 8, wherein the guide element further comprises at least one guiding section, wherein the at least one clamping section of the guide element is formed by the at least one second segment and the at least one guiding section of the guide element is formed by the at least one first segment, wherein the width of the at least one guiding section is greater than the diameter of the thread.

10. The fixation sleeve of claim 7, wherein each first segment and each second segment extends over the entire wall thickness, wherein the hardness of the material of the at least one first segment is greater than the hardness of the material of the at least one second segment.

11. The fixation sleeve of claim 7, wherein the elasticity of the material of the at least one second segment is greater than the elasticity of the at least one first segment and the wall thickness of the at least one second segment is greater than the wall thickness of the at least one first segment.

12. The fixation sleeve of claim Z, wherein the at least one first segment and the at least one second segment are arranged alternating in circumferential direction.

13. The fixation sleeve of claim 7, comprising at least one first element forming at least one first segment and a separately manufactured at least one second element forming at least one second segment, wherein the at least one first element is attached to the at least one second element

14. An assembly comprising a thread having a pre-defined diameter and a fixation sleeve according to claim 1.

15. An assembly comprising an implantable lead for a medical device and a fixation sleeve according to claim 1.
